(19) United States
(12) Patent Application Publication
(10) Pub. No.: US 2018/0008159 A1
(43) Pub. Date: Jan. 11, 2018

(54) SYSTEM AND METHOD OF REMOTE ECG MONITORING, REMOTE DISEASE SCREENING, AND EARLY-WARNING SYSTEM BASED ON WAVELET ANALYSIS

(71) Applicant: Fuzhou University, Fuzhou (CN)

(72) Inventors: Lianghung WANG, Fuzhou (CN); Minghui FAN, Fuzhou (CN)

(73) Assignee: Fuzhou University, Fuzhou (CN)

(21) Appl. No.: 15/677,056

(22) Filed: Aug. 15, 2017

Related U.S. Application Data
(63) Continuation-in-part of application No. PCT/CN2015/096538, filed on Dec. 7, 2015.

(30) Foreign Application Priority Data
Nov. 5, 2015 (CN) 201510742075.1

(51) Int. Cl. A61B 5/04; A61B 5/0245; A61B 5/044; A61B 5/0432; A61B 5/046; A61B 5/00

(52) U.S. Cl. CPC A61B 5/04018; A61B 5/046; A61B 5/746; A61B 5/7232; A61B 5/6831; A61B 5/6823; A61B 5/0006; A61B 5/044; A61B 5/0432; A61B 5/0245; A61B 5/0022; A61B 2560/0214

(57) ABSTRACT

The invention relates to the system and method of remote ECG monitoring, remote disease screening, and early-warning system based on wavelet analysis. The system includes a wireless ECG signal acquisition device, a mobile terminal, and a cloud storage platform. The wireless ECG signal acquisition device worn on the user's chest is used to collect ECG signals anywhere and anytime. The method includes transmitting the ECG signals to the mobile terminal using the wavelet analysis algorithm, analyzing and processing the received ECG signal, and uploading the processed ECG signals to the cloud storage platform. The cloud storage platform stores users' personal information and ECG signals. According to the ECG features detection with support vector machine learning algorithm for heart diseases diagnosis and features classification, the system gives feedback report and proposal, and transmits them to the mobile terminal.

### CROSS-REFERENCE TO RELATED APPLICATIONS

This application is a Continuation-in-Part of International Application No. PCT/CN2015/096538 filed on Dec. 7, 2015, which claims priority from Chinese patent Application No. 201510742075.1, filed on Nov. 5, 2015, the entire contents of which are incorporated herein by reference.

### TECHNICAL FIELD

The invention relates to ECG physiological information acquisition and monitoring, particularly to a remote ECG monitoring, remote disease screening, and early-warning system based on wavelet analysis.

### BACKGROUND

Cardiovascular disease is a serious problem faced by human beings. Such kind of diseases are often sudden or acute. Lives of patients will be threatened if a golden treatment period is not caught. Therefore, more and more patients need monitoring, recording, and analyzing of their cardiac signal anytime anywhere.

Cardiovascular and cerebrovascular diseases are common diseases for the elders. As the aging of the society is getting sever, the number of empty nesters is increasing, who usually lack timely treatment. For the elders who suffer from heart disease, how to get guardianship and early warning at the first time is critical. Accordingly, portable monitoring system is required which has a simple operation and a real-time monitoring.

Meanwhile, given the economic development and the changing of lifestyle, the chance for exercising is reduced, the diet is refined, and the living pressure is increased. Thus, cardiovascular and cerebrovascular diseases are gradually spreading among middle-aged people and matured people, Even worse, some people got attacked by the disease at their 30s. Huge losses are caused for the family, the company, and the society. Therefore, monitoring, recording, and analyzing the heart for middle-aged and matured population are necessary.

Currently, the wireless ECG physiological information acquisition system needs a complex mechanical support system, limiting the user's movements. A fixed electrode acquisition system is used because the cohesive fixed electrode can cause discomfort, skin allergies, and other adverse symptoms if the user wears it for a prolonged period. The traditional wireless ECG physiological information acquisition system does not have an intelligent terminal. Users must have a certain medical knowledge to use the device given its lack of a human-computer interaction module. Moreover, a traditional system can only give real-time ECG signal, and only doctors can identify the disease. First, the date is uploaded to a large data analysis platform, The data analysis results are transmitted back to the user. Not only this is time consuming, but also storage, analysis, and the complex signal processing functions are required. Moreover, feature points and eigenvalue of the signal cannot be recognized. Further, the screening and early warning information cannot be provided based on the recognition of the signal.

### SUMMARY

Thus, the invention aims to provide a remote ECG monitoring, and remote disease screening and early-warning system and method, based on wavelet analysis, and to overcome the shortcomings of the traditional acquisition system of ECG signal. The system of remote ECG monitoring, and early-warning based on wavelet analysis and ultra-low power Bluetooth technology is used to monitor and alert heart signal.

The invention adopts the following solutions. A remote ECG monitoring, and remote disease screening and early-warning system and method, based on a wavelet analysis, includes a wireless ECG signal acquisition device, a mobile terminal, and a cloud storage platform. The wireless ECG signal acquisition device is configured to be worn on a user's chest. The wireless ECG signal acquisition device is configure to collect ECG signal in real time and transmit the ECG signal to the mobile terminal. The mobile terminal is configured to analyze and process the received ECG signal using a wavelet analysis algorithm. The mobile terminal is configured to upload the processed ECG signal to the cloud storage platform. The cloud storage platform is configured to store the user's personal information, the ECG signal, waveform features of the ECG signal obtained by analysis, and a type of heart disease. The system is configured to determine whether the user's heart is healthy or what kind of disease the user is suffering from according to the waveform features of the ECG signal using a support vector machine based heart disease diagnosis algorithm. The system is configured to provide the user with heart disease screening recommendation. The system is configured to obtain a health and recovery condition of the user's heart by comparing the ECG signal collected by the wireless ECG signal acquisition device with a historically stored ECG signal. The system is configured to inform the user about the health and recovery condition of the user's heart.

Further, the wireless ECG signal acquisition device is a wireless wearable cardiovascular signal acquisition sensor. The sensor includes an ECG signal acquisition patch, an ECG signal acquisition analog circuit, a digital processing circuit, a low-power Bluetooth transmission circuit, and a rechargeable power supply circuit. An output terminal of the ECG signal acquisition patch is connected to an input terminal of the ECG signal acquisition analog circuit. An output terminal of the ECG signal acquisition analog circuit is connected to an input terminal of the digital processing circuit. An output terminal of the digital processing circuit is connected to the low-power Bluetooth transmission circuit. The low-power Bluetooth transmission circuit is configured to transmit the ECG signal to the mobile terminal, The ECG signal acquisition patch, the ECG signal acquisition analog circuit, the digital processing circuit, and the low-power Bluetooth transmission circuit are all connected to the rechargeable power supply circuit.

Further, the mobile terminal includes a low-power Bluetooth receiving circuit, a wavelet algorithm analysis module, an application client module, a data storage module, a display module, and an alarm module. The low-power Bluetooth receiving circuit is configured to receive the ECG signal transmitted from a low-power Bluetooth transmission circuit. An output terminal of the low-power Bluetooth receiving circuit is connected to an input terminal of the wavelet algorithm analysis module. The wavelet algorithm analysis module is configured to analyze the received ECG signal to obtain the waveform features of the ECG signal. An output terminal of the wavelet algorithm analysis module is connected to the application client module and the data storage module. The data storage module is configure to store the ECG signal and the waveform features of the ECG signal obtained by analysis. The application client module is connected to the display module and the alarm module. The display is configured to present the ECG signal and the waveform features of the ECG signal obtained by analysis. The alarm module is configured to send an alarm when the ECG signal of the user is abnormal.

Further, the cloud storage platform includes a low-power wireless receiving circuit, a big data cloud storage module, a support vector machine based heart disease diagnosis algorithm module, and a feedback report and proposal plan module. The low-power wireless receiving circuit is configured to receive the ECG signal transmitted by a transmission circuit of the mobile terminal module. An output terminal of the low-power wireless receiving circuit is connected to an input terminal of the big data cloud storage module. The big data cloud storage module is configured to store the ECG signal, the waveform features of the ECG signal obtained by analysis, and heart disease features of the user. An output terminal of the big data cloud storage module is connected to an input terminal of the support vector machine based heart disease diagnosis algorithm module. The support vector machine based heart disease diagnosis algorithm module is configured to determine the waveform features of the ECG signal based on the ECG signal received by the big data cloud storage module. The support vector machine based heart disease diagnosis algorithm module is configured to determine whether the user's heart is healthy or what kind of disease the user is suffering from. The output terminal of the support vector machine based heart disease diagnosis algorithm module is connected to an input terminal of the feedback report and proposal plan module, then send the report to the mobile terminal module.

Further, the wireless wearable cardiovascular signal acquisition sensor is fixed on the user's chest with an elastic bandage.

Further, the rechargeable power supply circuit includes a lithium battery. The lithium battery is configured to supply power to the ECG signal acquisition patch, an ECG signal acquisition analog circuit, a digital processing circuit, and a low-power Bluetooth transmission circuit. The lithium battery is a rechargeable battery.

Further, the wireless wearable cardiovascular signal acquisition sensor further includes a notch filter circuit. The notch filter circuit is configured to remove AC frequency interference of 50 Hz.

Further, the digital processing circuit includes a compression algorithm of the ECG signal. The digital processing circuit is configured to compress a great amount of digitalized ECG signal to reduce the transmission loss rate and transmission power of the low-power Bluetooth transmission circuit.

Further, the mobile terminal further includes a short message transmission module. The short message transmission module is configured to send the ECG signal and the waveform features of the ECG signal obtained by analysis to the user's family and a doctor at the hospital.

The personal information includes the user's name, gender, age, height, weight, medication history, and family contact.

Further, the mobile terminal is a smart phone.

The invention also adopts the following solutions.

A method of remote ECG monitoring, remote disease screening, and early-warning based on wavelet analysis, include the following steps:

Step S1: collecting the ECG signal in real time after the user wears the wireless ECG signal acquisition device on the user's chest;

Step S2: transmitting the collected ECG signal by an ECG signal acquisition patch in the wireless ECG signal acquisition device through an analog circuit and a digital processing circuit containing a compression algorithm to a low-power Bluetooth transmission circuit; and transmitting the collected ECG signal by the low-power Bluetooth transmission circuit to the mobile terminal;

Step S3: receiving the ECG signal by a low-power Bluetooth receiving circuit in the mobile terminal from the low-power Bluetooth transmission circuit; and transmitting the ECG signal to the wavelet analysis algorithm module for analysis and processing;

Step S4: processing the received ECG signal by a wavelet analysis algorithm module using the wavelet analysis algorithm: detecting each peak point of the ECG signal; calculating the time of each peak interval to obtain waveform features of the ECG signal; transmitting data and the waveform features of the ECG signal by the wave analysis algorithm module to the application client module;

Step S5: establishing the user's personal account by the application client module; controlling the display module through the application client module to display the data and the waveform of the ECG signal obtained by wavelet analysis; sending an alarm by the alarm module controlled by the application client module when the user's ECG signal is significantly abnormal;

Step S6: uploading the processed ECG signal by the application client module to the cloud storage platform; aggregating and storing the user's personal information, the ECG signal, and the waveform features of the ECG signal obtained by analysis, by the cloud storage platform; classifying each ECG waveform by the cloud storage platform using the support vector machine based cardiac diagnosis algorithm and a heart rate classification model; wherein classifications that can be realized include atrial premature beat, atrial fibrillation, atrial premature beat, ventricular flutter, atrial flutter, and normal heart rate;

Step S7: generating an analysis report by the cloud storage platform when an abnormal heart rate is found; transmitting an ECG signal waveform of the abnormal heart rate and a heart rate classification to the application client module; feeding back the ECG signal waveform of the abnormal heart rate and the heart rate classification to the user; and exporting the report by the user to directly show the report to the doctor; and

Step S8: modifying the heart rate classification by the doctor on the cloud storage platform when a judgment of the heart rate classification model is wrong; memorizing the ECG data by the classification model; readjusting parameters of the heart rate classification model; and establishing a specific classification model for each user by the cloud platform.

The invention constructs a portable electronic and network system, which combines the three techniques of ECG signal acquisition and processing, network service, and back-end platform monitoring. On the one hand the ECG signal acquisition patch is fixed on the user's chest with an elastic bandage. The system has no influence on the normal activities of the human body and reduces discomfort. On the other hand, the most common portable product is the mobile phone. The mobile terminal transfer station is widely used through a mobile phone. Furthermore, the system is portable and reduces equipment costs. Thus, the system can serve more users. The application client and cloud storage platform is provided in the mobile terminal. The mobile terminal analyzes all kinds of heart diseases and transmits an ECG signal waveform of the abnormal heart rate and a heart rate classification to the application client. Users can manage their individual accounts through a simple interface of the application easily.

Compared with the existing technology, the invention adopts the system of remote ECG monitoring and early-warning based on wavelet analysis, Ultra-low power Bluetooth technology is used to monitor and alert heart signal. This system provides a wearable acquisition patch, which can be worn at work or while playing leisure sports, The system can collect real-time ECG signal, The ECG signal is transmitted to the mobile terminal through wireless Bluetooth technology. The mobile terminal can save the ECG signal. Heart disease features are analyzed by the wavelet analysis algorithm module, which is set up in the mobile phone. The ECG signal and the analysis data are displayed in the application client. The single lead ECG signal can show the physical information, The waveform features of ECG signal can be obtained by using a complex and efficient wavelet analysis algorithm. Then, each ECG waveform is categorized by the cloud storage platform using the support vector machine based cardiac diagnosis algorithm and a heart rate classification model. The signal type will be determined if the ECG signal is abnormal. Whether the early warning information should be issued is determined according to information such as user's gender and age, so as to remind the user to take appropriate measures. Moreover, the user can control the specific function by using the application client, transmit data to the family or doctor by using the SMS or online mode, and achieve a remote monitoring function. The user can also transmit data to the cloud storage platform, collect data, borrow big data analysis on the development of disease, and forward early screening recommendations, Doctors can also use telnet and cloud storage platform to access screening data, research, and propose the corresponding treatment methods.

### BRIEF DESCRIPTION OF THE DRAWINGS

FIG. 1 shows the overall system architecture.

FIG. 2 shows the schematic diagram of the system.

FIG. 3 shows the functional block diagram of the system.

FIG. 4 shows the schematic diagram of the wavelet analysis algorithm.

FIG. 5 shows the schematic diagram of support vector machine based heart disease diagnosis algorithm.

FIG. 6 shows the function framework of the application client.

### DETAILED DESCRIPTION OF THE INVENTION

The following Figures and examples further describe the invention.

This embodiment provides a system remote ECG monitoring, and remote disease screening and early-warning based on wavelet analysis, which includes a wireless ECG signal acquisition device, a mobile terminal, and a cloud storage platform. The wireless ECG signal acquisition device is configured to be worn on a user's chest. The wireless ECG signal acquisition device is configured to collect ECG signal in real time and transmit the ECG signal to the mobile terminal. The mobile terminal is configured to analyze and process the received ECG signal using a wavelet analysis algorithm. The mobile terminal is configured to upload the processed ECG signal to the cloud storage platform. The cloud storage platform is configured to store the user's personal information, disease history, the ECG signal, waveform features of the ECG signal obtained by analysis, and a type of heart disease. The mobile terminal is a smart phone.

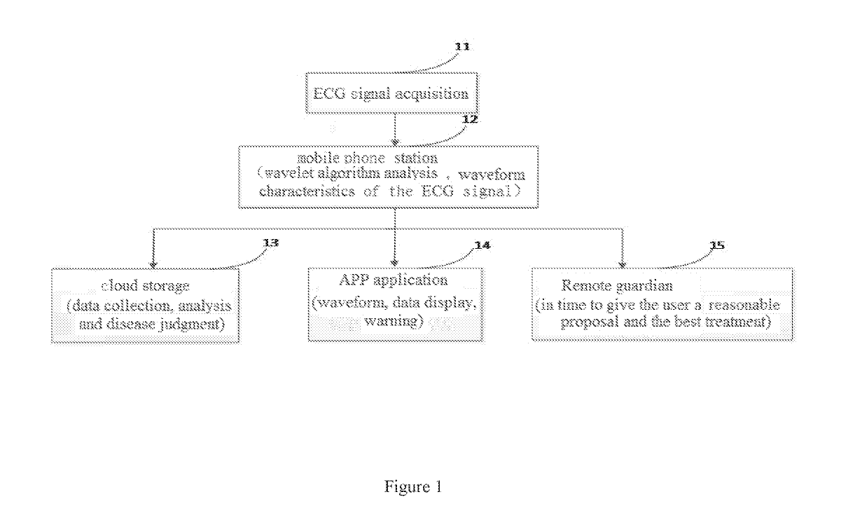

In this embodiment, FIG. 1 shows that the system provides a wearable acquisition patch 11, which can be worn at work, leisure, or while playing sports. The system can collect ECG signal through the wireless Bluetooth technology, transmit the ECG signal data to the mobile phone terminal 12, and analyze and process the received ECG signal using the wavelet analysis algorithm implanted in the mobile phone. The application in the mobile phone terminal 14 displays the ECG signal and the data obtained by analysis. The wavelet analysis algorithm is used to create a preliminary judgment of the ECG signal waveform features of the tested person. The corresponding alarm signal is issued if abnormal ECG signal is detected. The user can use the APP function through the control button on the application 14. Through SMS or the Internet, the user can transmit the data to the remote guardian 15, such that the remote monitoring function is achieved. The data can be transmitted to the cloud storage platform 13. Each ECG waveform is categorized by the cloud storage platform using the support vector machine based cardiac diagnosis algorithm and a heart rate classification model. The doctor can analyze the collected ECG signal through the cloud storage platform and understand the user's heart condition in real time. Obtaining the previous ECG signal data and identifying the heart disease condition before treatment enable the doctor to advise, help, and treat users well. Each user's data can be gathered to form a large database. Thus, this terminal allows researchers to request for data and perform a large data analysis on the development trend of the disease, in order to search for an improved treatment.

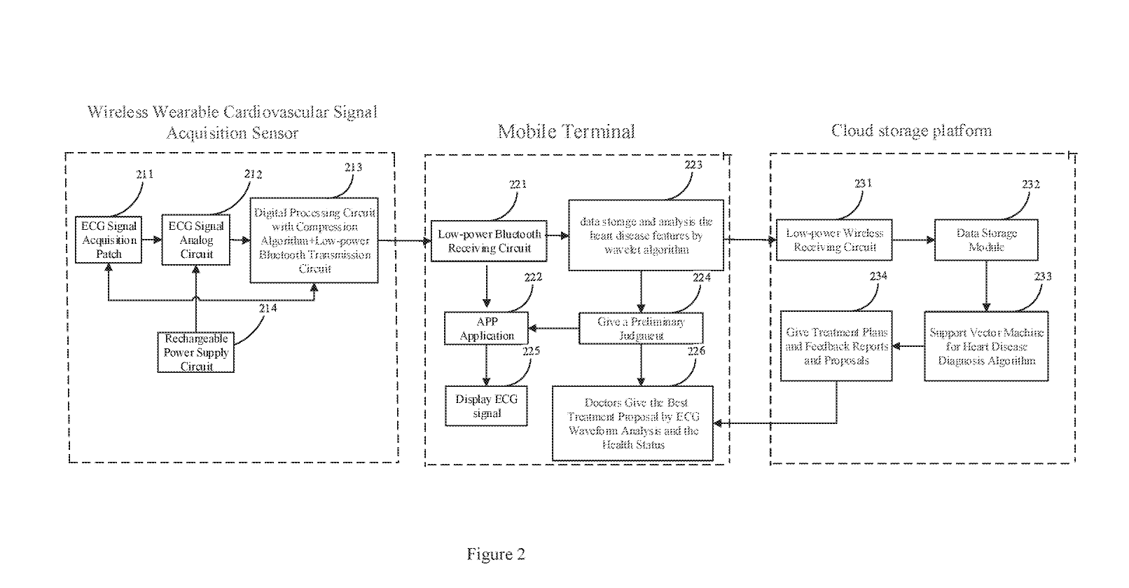

In this embodiment, FIG. 2 shows that the wireless ECG signal acquisition device is a wireless wearable cardiovascular signal acquisition sensor. The sensor includes an ECG signal acquisition patch 211, an ECG signal acquisition analog circuit 212, a digital processing circuit 213, a low-power Bluetooth transmission circuit 213 and a rechargeable power supply circuit 214. An output terminal of the ECG signal acquisition patch is connected to an input terminal of the ECG signal acquisition analog circuit. An output terminal of the ECG signal acquisition analog circuit is connected to an input terminal of the digital processing circuit. An output terminal of the digital processing circuit is connected to the low-power Bluetooth transmission circuit. The low-power Bluetooth transmission circuit is configured to transmit the ECG signal to the mobile terminal. The ECG signal acquisition patch, the ECG signal acquisition analog circuit, the digital processing circuit, and the low-power Bluetooth transmission circuit are all connected to the rechargeable power supply circuit.

In this embodiment, the wireless wearable cardiovascular signal acquisition sensor is fixed on the user's chest with an elastic bandage.

In this embodiment, the rechargeable power supply circuit includes a lithium battery. The lithium battery is configured to supply power to the ECG signal acquisition patch, an ECG signal acquisition analog circuit, a digital processing circuit, and a low-power Bluetooth transmission circuit. The lithium battery is a rechargeable battery. The power is supplied by a thin and rechargeable lithium battery so as to reduce the purchase cost of battery. The battery can be recharged any time, such that the operating time of the system is prolonged.

In this embodiment, the wireless wearable cardiovascular signal acquisition sensor further includes a notch filter circuit. The notch filter circuit is configured to remove AC frequency interference of 50 Hz.

In this embodiment, the wavelet analysis algorithm is realized as shown in FIG. 4. Based on the principle of analyzing and processing the ECG signal, the wavelet analysis algorithm module is provided in the mobile phone. The ECG signal 41 is reviewed. The wavelet analysis algorithm module can detect the ECG signal of each peak point, calculate the time interval 42, analyze the morphology of ECG signal 43, and finally obtain the corresponding values. If the ECG signal is abnormal, the system automatically identifies severely abnormal ECG signal to perform early warning action. Advice and feedback 44 will be provided. The application client controls the module that displays the data obtained by wavelet analysis, which allows users to derive an enhanced intuitive understanding of their ECG signal information.

In this embodiment, the implementation of the support vector machine for heart disease diagnosis algorithm is shown in FIG. 5. Based on the principle of analyzing and the processing of ECG signal, the support vector machine based heart disease diagnosis algorithm module is provided in the cloud storage platform. ECG signal 51 is received. The support vector machine based heart disease diagnosis algorithm module can categorize the features of each ECG signal waveform 52 and analyze the morphology of ECG signal. The classification can be diagnosed as atrial premature beat, atrial fibrillation, atrial premature beat, ventricular flutter, atrial flutter, and normal heart rate 53. The corresponding heart disease could be diagnosed finally. If an abnormal rhythm is found, the cloud storage platform generates an analysis report, and transmits the abnormal ECG signal waveform and cardiac rhythm categories to the application client module and sends feedback to the user 54. The user can understand her ECG information and directly export the report to show the doctor.

In this embodiment, FIG. 2 shows that the mobile terminal includes a low-power Bluetooth receiving circuit 221, data storage and wavelet algorithm analysis module 223, application client module 222, preliminary judgment module 224, display module 225, and alarm module 226. The low-power Bluetooth receiving circuit is configured to receive the ECG signal transmitted from a low-power Bluetooth transmission circuit. An output terminal of the low-power Bluetooth receiving circuit is connected to an input terminal of the wavelet algorithm analysis module. The wavelet algorithm analysis module is configured to filter the received signal to exclude the ECG baseline drift, power frequency interference, and EMG interference. The waveform features of the ECG signal are obtained and analyzed. Whether the ECG signal is normal is determined. An output terminal of the wavelet algorithm analysis module is connected to the application client module and the data storage module. The data storage module is configured to store the ECG signal and the waveform features of the ECG signal obtained by analysis. The application client module is connected to the display module and the alarm module. The display is configured to present the ECG signal and the waveform features of the ECG signal obtained by analysis. The alarm module is configured to send an alarm when the ECG signal of the user is abnormal.

In this embodiment, FIG. 2 shows that the cloud storage platform includes a low-power wireless receiving circuit 231, data storage module 232, support vector machine based heart disease diagnosis algorithm module 233, and feedback report and proposals module 234. The low-power wireless receiving circuit receives the ECG signal of the mobile terminal transmission circuit. The output terminal of such circuit is connected to the data storage module. The data storage module is used for storing the ECG signal. The support vector machine based heart disease diagnosis algorithm module is connected to the data storage module. The waveform features of ECG signal and the features of the user's heart disease are analyzed to determine whether the user's heart is healthy or what kind of disease the user is suffering. The user's heart disease screening reference recommendations are fed back to the mobile terminal module through a report. The user can know the information of the heart.

In this embodiment, since the ECG signal is used as the collected data mainly, through a single channel and multi-channel acquisition technology, the ECG physiological signal acquisition is completed. The physiological signal is sent to the network transfer station that contains the functions of encryption and decryption, data compression, simple media access, RISC processor, wireless transmission, and reception in low power wireless Bluetooth technology. The mobile phone is the most popular portable product. Thus, the mobile phone is used as the network transfer station. The capacity the memory of the mobile phone is high and can store ECG signal. The wavelet analysis algorithm provided in the mobile phone application can judge the extracted signal and provide the corresponding early warning information.

In this embodiment, the digital processing circuit includes a compression algorithm of the ECG signal. The digital processing circuit is configured to compress a great amount of digitalized ECG signal to reduce the transmission loss rate and transmission power of the low-power Bluetooth transmission circuit.

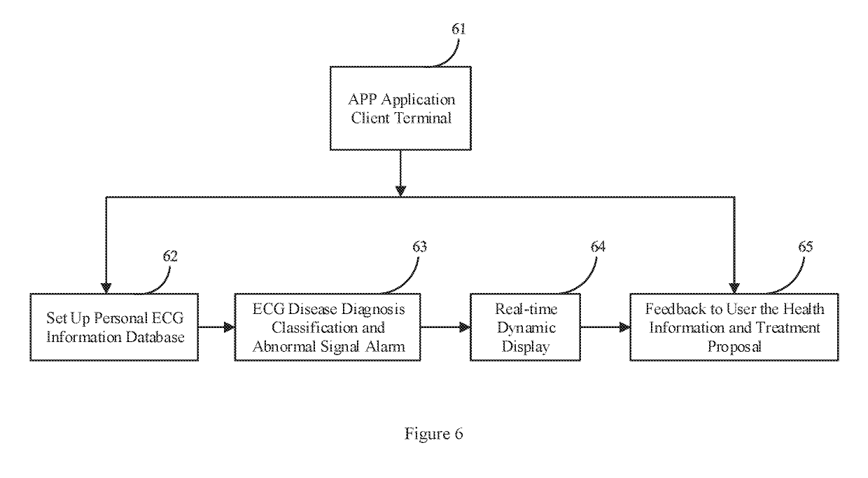

In this embodiment, the application client module is used for controlling the module that displays ECG signal and the wavelet algorithm analysis module to obtain the waveform features of ECG signal. The alarm module is controlled to send an alarm when the ECG signal is abnormal. The application client module can also be used to establish the user's personal account and to set up personal information. The personal information includes the user's name, gender, age, height, weight, medication history, and family contact. FIG. 6 shows the client side of the application framework. The user can use the application to conduct various types of operations. In the client application, the users can set up personal accounts 62 and set up the personal information, such as name, gender, age, height, weight, medication history, and family contact. The personal account can be used to save the user's collection records and analysis records. Thus, users can see their own historical records and manage their personal accounts easily. The current acquisition of the ECG signal and the data analysis through the real-time dynamic of the application can be presented through the display interface of the display module 64. The exception of the alarm 63 is activated immediately to remind users if the analysis results of the signal are abnormal. Users can obtain the results of the preliminary screening and understand the information report of their own heart health 65.

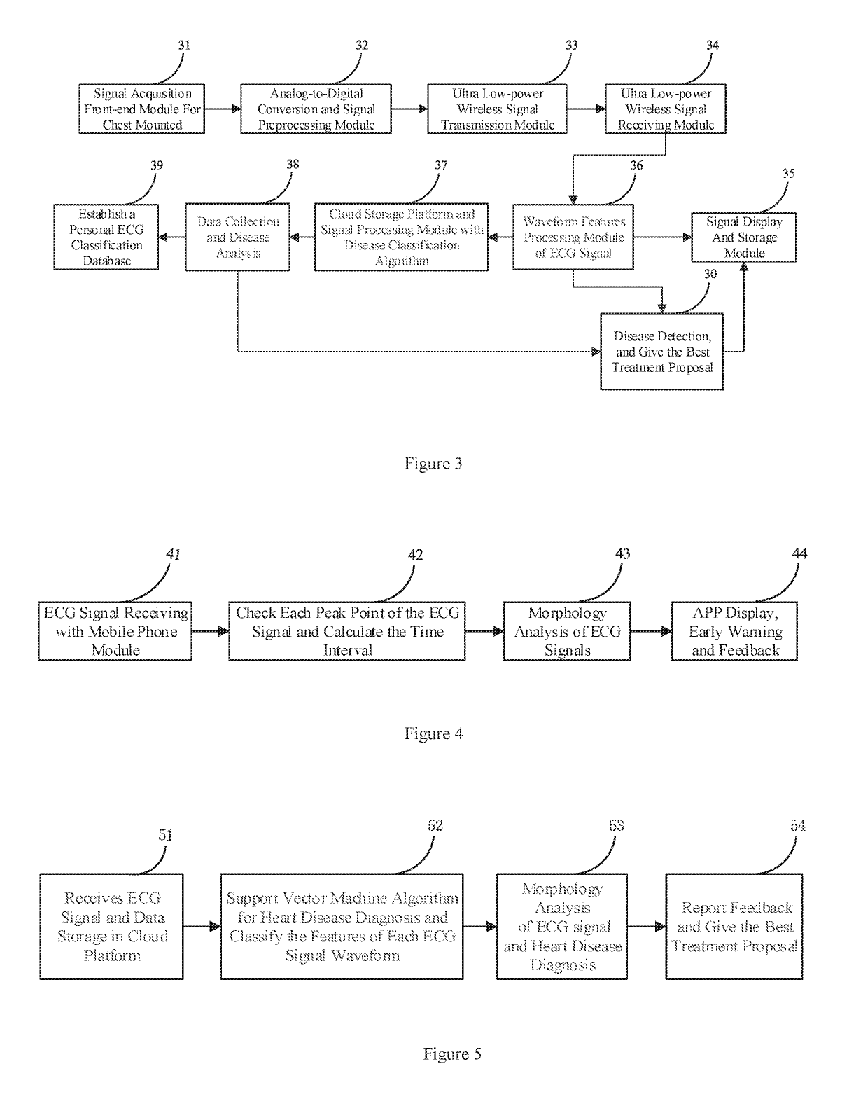

In this embodiment, FIG. 3 shows that the system can be described according to the functional diagram. The system through the chest mounted front acquisition module 31 collects human heart ECG signal. Using a single lead method, the standard single lead ECG signal on the chest is collected. The detection system must be worn for a long time. We must consider low power consumption in addition to low noise and high performance. The external circuit demands other considerations, such as a notch filter, which is mainly used for filtering out the AC frequency interference of the 50 Hz. Given that every kind of signal interference is available during use, the human body is a large antenna. The 50 Hz AC signal is coupled to the human body. The noise source passing through the human body produces strong interference on the detection circuit when the ECG physiological signal is weak. The ECG signal is almost impossible to be detected as long as a noise source exists. Therefore, the design and application of the notch filter circuit are critical.

Analog-to-digital conversion and signal preprocessing module 32 can handle the collected signal. The ECG signal is input to the signal amplification module. The signal is amplified and filtered such that the signal is easy to be processed subsequently. The amplified signal goes through digital and low power processing and is converted to RF signal, which is easy to transmit. The adjustable medical instrument system can be achieved by reducing the size, power consumption, and overall cost.

The ultra-low power wireless signal transmission module 33 is compatible with ultra-low power Bluetooth protocol 4.0, which includes the microprocessor core with high performance and low power consumption and can be used as the controller of the transmission. Ultra-low power wireless signal receiving module 34 is the mobile terminal receiving module. Users can receive the monitoring signal on the mobile phones 221 using Bluetooth 4.0, and wait for the analysis and processing of the algorithm. The signal is processed by the early warning algorithm module 36. Based on the wavelet analysis algorithm, the early warning algorithm module filters the ECG signal and extracts the feature extraction and feature information of the ECG signal. The judgment mechanism is established according to different age and sex groups. Whether the extracted signal is normal is determined. And Provides the corresponding early warning information is provided. The detection of singular points of the ECG signal is realized. Display and storage module 35 can save users' ECG signal. The acquired ECG signal and data obtained by the analysis algorithm can be displayed by mobile phone applications 222 in real time. The function can be directly provided for the user. The collected waveforms is displayed in real time. Timely alarm can be made. The intelligent terminal, wireless receiving, and signal processing modules together make up a program with a visual interface on the terminal. The collected waveform of the ECG signal is displayed. Other physiological information reflected by the calculated heart rate and ECG are given.

Finally, cloud storage platform 37 analyzes the waveform features of the ECG signal and features of user heart disease, as well as collects and stores the data of all users. Professionals can compare and monitor the cases in an area through data analysis and monitor a single case in a long term. Thus, studying the long-term development trend and group development trend of the symptom is possible. The doctor can also monitor the terminal user's heart condition to provide better services. The data collection and analysis 38 can provide abundant data and help doctors control the overall trend of the disease. The research of treatment of the researchers is facilitated.

The discussion above illustrates the invention, equivalent modification, and alternations within the scope of the invention patent.

What is claimed is:

1. A system of remote ECG monitoring, remote disease screening, and early-warning based on wavelet analysis, comprising:
a wireless ECG signal acquisition device;
a mobile terminal; and
a cloud storage platform;
wherein
the wireless ECG signal acquisition device is configured to be worn on a user's chest;
the wireless ECG signal acquisition device is configured to collect ECG signal in real time and transmit the ECG signal to the mobile terminal;
the mobile terminal is configured to analyze and process the received ECG signal using a wavelet analysis algorithm;
the mobile terminal is configured to upload the processed ECG signal to the cloud storage platform;
the cloud storage platform is configured to store the user's personal information, the ECG signal, waveform features of the ECG signal obtained by analysis, and a type of heart disease;
the system is configured to determine whether the user's heart is healthy or what kind of disease the user is suffering from according to the waveform features of the ECG signal using a support vector machine based heart disease diagnosis algorithm;
the system is configured to provide the user with heart disease screening recommendation;
the system is configured to obtain a health and recovery condition of the user's heart by comparing the ECG signal collected by the wireless ECG signal acquisition device with a historically stored ECG signal; and
the system is configured to inform the user about the health and recovery condition of the user's heart.

2. The system of remote ECG monitoring, remote disease screening, and early-warning based on wavelet analysis of claim 1, wherein
the wireless ECG signal acquisition device is a wireless wearable cardiovascular signal acquisition sensor;
the wireless wearable cardiovascular signal acquisition sensor includes
an ECG signal acquisition patch,
an ECG signal acquisition analog circuit,
a digital processing circuit,
a low-power Bluetooth transmission circuit, and
a rechargeable power supply circuit;
wherein
an output terminal of the ECG signal acquisition patch is connected to an input terminal of the ECG signal acquisition analog circuit;
an output terminal of the ECG signal acquisition analog circuit is connected to an input terminal of the digital processing circuit;
an output terminal of the digital processing circuit is connected to the low-power Bluetooth transmission circuit;
the low-power Bluetooth transmission circuit is configured to transmit the ECG signal to the mobile terminal; and
the ECG signal acquisition patch, the ECG signal acquisition analog circuit, the digital processing circuit, and the low-power Bluetooth transmission circuit are all connected to the rechargeable power supply circuit.

3. The system of remote ECG monitoring, remote disease screening, and early-warning based on wavelet analysis of claim 1, wherein the mobile terminal includes
a low-power Bluetooth receiving circuit,
a wavelet algorithm analysis module,
an application client module,
a data storage module,
a display module, and
an alarm module;
wherein
the low-power Bluetooth receiving circuit is configured to receive the ECG signal transmitted from a low-power Bluetooth transmission circuit;
an output terminal of the low-power Bluetooth receiving circuit is connected to an input terminal of the wavelet algorithm analysis module;
the wavelet algorithm analysis module is configured to analyze the received ECG signal to obtain the waveform features of the ECG signal;
an output terminal of the wavelet algorithm analysis module is connected to the application client module and the data storage module;
the data storage module is configure to store the ECG signal and the waveform features of the ECG signal obtained by analysis;
the application client module is connected to the display module and the alarm module;
the display is configured to present the ECG signal and the waveform features of the ECG signal obtained by analysis;
the alarm module is configured to send an alarm when the ECG signal of the user is abnormal;
the application client module is configured to control the display module to show the ECG signal and the waveform features of the ECG signal obtained by the wavelet algorithm analysis module, and the application client module is configured to control the alarm module to generate an alarm when the ECG signal is abnormal; and
the application client module is configured to establish the user's personal account and personal information.

4. The system of remote ECG monitoring, remote disease screening, and early-warning based on wavelet analysis of claim 1, wherein the cloud storage platform includes
a low-power wireless receiving circuit,
a big data cloud storage module,
a support vector machine based heart disease diagnosis algorithm module, and
a feedback report and proposal plan module;
wherein
the low-power wireless receiving circuit is configured to receive the ECG signal transmitted by a transmission circuit of the mobile terminal module;
an output terminal of the low-power wireless receiving circuit is connected to an input terminal of the big data cloud storage module;
the big data cloud storage module is configured to store the ECG signal, the waveform features of the ECG signal obtained by analysis, and heart disease features of the user;
an output terminal of the big data cloud storage module is connected to an input terminal of the support vector machine based heart disease diagnosis algorithm module;
the support vector machine based heart disease diagnosis algorithm module is configured to determine the waveform features of the ECG signal based on the ECG signal received by the big data cloud storage module, and the support vector machine based heart disease diagnosis algorithm module is configured to determine whether the user's heart is healthy or what kind of disease the user is suffering from;
the output terminal of the support vector machine based heart disease diagnosis algorithm module is connected to an input terminal of the feedback report and proposal plan module;
the feedback report and proposal plan module is configured to provide a recommendation regarding the user's heart disease screening; and
the feedback report and proposal plan module is configured to send the report to the mobile terminal module.

5. The system of remote ECG monitoring, remote disease screening, and early-warning based on wavelet analysis of claim 2, wherein the wireless wearable cardiovascular signal acquisition sensor is fixed on the user's chest with an elastic bandage.

6. The system of remote ECG monitoring, remote disease screening, and early-warning based on wavelet analysis of claim 2, wherein
the rechargeable power supply circuit includes a lithium battery;
the lithium battery is configured to supply power to the ECG signal acquisition patch, an ECG signal acquisition analog circuit, a digital processing circuit, and a low-power Bluetooth transmission circuit; and
the lithium battery is a rechargeable battery.

7. The system of remote ECG monitoring, remote disease screening, and early-warning based on wavelet analysis of claim 2, wherein
the wireless wearable cardiovascular signal acquisition sensor further includes a notch filter circuit; and
the notch filter circuit is configured to remove AC frequency interference of 50 Hz.

8. The system of remote ECG monitoring, remote disease screening, and early-warning based on wavelet analysis of claim 2, wherein
the digital processing circuit includes a compression algorithm of the ECG signal, and
the digital processing circuit is configured to compress a great amount of digitalized ECG signal to reduce the transmission loss rate and transmission power of the low-power Bluetooth transmission circuit.

9. The system of remote ECG monitoring, remote disease screening, and early-warning based on wavelet analysis of claim 1, wherein
the mobile terminal further includes a short message transmission module; and
the short message transmission module is configured to send the ECG signal and the waveform features of the ECG signal obtained by analysis to the user's family and a doctor at the hospital.

10. The system of remote ECG monitoring, remote disease screening, and early-warning based on wavelet analysis of claim 1, wherein
the personal information includes the user's name, gender, age, height, weight, medication history, and family contact.

11. The system of remote ECG monitoring, remote disease screening, and early-warning based on wavelet analysis of claim 3, wherein
the personal information includes the user's name, gender, age, height, weight, medication history, and family contact.

12. A method of remote ECG monitoring, remote disease screening, and early-warning based on wavelet analysis, comprising the following steps:
Step S1: collecting the ECG signal in real time after the user wears the wireless ECG signal acquisition device on the user's chest;
Step S2: transmitting the collected ECG signal by an ECG signal acquisition patch in the wireless ECG signal acquisition device through an analog circuit and a digital processing circuit containing a compression algorithm to a low-power Bluetooth transmission circuit; and transmitting the collected ECG signal by the low-power Bluetooth transmission circuit to the mobile terminal;
Step S3: receiving the ECG signal by a low-power Bluetooth receiving circuit in the mobile terminal from the low-power Bluetooth transmission circuit; and transmitting the ECG signal to the wavelet analysis algorithm module for analysis and processing;
Step S4: processing the received ECG signal by a wavelet analysis algorithm module using the wavelet analysis algorithm: detecting each peak point of the ECG signal; calculating the time of each peak interval to obtain waveform features of the ECG signal; transmitting data and the waveform features of the ECG signal by the wavelet analysis algorithm module to the application client module;
Step S5: establishing the user's personal account by the application client module; controlling the display module through the application client module to display the data and the waveform of the ECG signal obtained by wavelet analysis; sending an alarm by the alarm module controlled by the application client module when the user's ECG signal is significantly abnormal;
Step S6: uploading the processed ECG signal by the application client module to the cloud storage platform; aggregating and storing the user's personal information, the ECG signal, and the waveform features of the ECG signal obtained by analysis, by the cloud storage platform; classifying each ECG waveform by the cloud storage platform using the support vector machine based cardiac diagnosis algorithm and a heart rate classification model; wherein classifications that can be realized include atrial premature beat, atrial fibrillation, atrial premature beat, ventricular flutter, atrial flutter, and normal heart rate;
Step S7: generating an analysis report by the cloud storage platform when an abnormal heart rate is found; transmitting an ECG signal waveform of the abnormal heart rate and a heart rate classification to the application client module; feeding back the ECG signal waveform of the abnormal heart rate and the heart rate classification to the user; and exporting the report by the user to directly show the report to the doctor; and
Step S8: modifying the heart rate classification by the doctor on the cloud storage platform when a judgment of the heart rate classification model is wrong; memorizing the ECG data by the classification model; readjusting parameters of the heart rate classification model; and establishing a specific classification model for each user by the cloud platform.
